(19) United States
(12) Patent Application Publication
(10) Pub. No.: US 2005/0203558 A1
(43) Pub. Date: Sep. 15, 2005

(54) Device for applying and monitoring medical rotablation

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/058,655

(22) Filed: Feb. 15, 2005

(30) Foreign Application Priority Data
Feb. 20, 2004 (DE) 10 2004 008 368.1

(51) Int. Cl. A61B 17/14; A61B 17/32; A61B 8/00; A61B 8/12; A61B 8/14

(52) U.S. Cl. 606/180

(57) ABSTRACT

Arrangement for executing and monitoring rotablation, in which a rotating drill head arranged at the tip of a catheter removes plaque adhering to a vessel wall while pushing aside normal vessel tissue, with a rotablation catheter and an IVUS catheter being integrated into one constructional unit.

### CROSS REFERENCE TO RELATED APPLICATIONS

This application claims priority to the German application No. 10 2004 008 368.1, filed Feb. 20, 2004 which is incorporated by reference herein in its entirety.

### FIELD OF INVENTION

The invention relates to a device for executing and monitoring rotablation, in which a rotating drill head arranged at the tip of a catheter removes plaque attached to the vessel wall while pushing aside normal tissue.

### BACKGROUND OF INVENTION

One of the most frequent diseases in the world with fatal consequences is vascular artery disease, especially a heart attack. This is caused by arthoscelerosis. In such cases build-ups (atherosclerotic plaque) result in a “blockage of the coronary arteries. When coronary angiography shows major narrowing (stenoses) in the coronary blood vessel which cause angina pectoris, restrict capabilities and/or threaten the patient, then currently in the majority of cases a PTCA (Percutaneous transluminal coronary angioplasty) is undertaken. This involves widening the narrowed points of the coronary arteries with what are known as “balloon catheters”.

Clinical studies have shown that with this method a restenosis can occur in many patients, in some case up to 50% of the patients show restenosis. An alternative method of plaque removal has thus started to be widely used in recent years, known as high-frequency rotablation angioplasty, which offers advantages, especially with heavily fibrotic or calcified and/or extensive stenoses.

Coronary rotablation angioplasty is what is known as a “debulking” system (recanalization of stenosized coronary arteries).

The rotablation angioplasty system consists of a diamond tipped drill head rotating at high speed which selectively removes calcified and fibrotic plaques, while the normal elastic vessel wall is pushed aside from the drill head and is not damaged (“differential cutting”). The microparticles produced are rinsed away in the periphery. The method has established itself as a valuable instrument for heavily calcified lesions which cannot be removed by simple balloon angioplasty. By contrast with balloon angioplasty the stenosis is not widened. At a typical rotational speed of 150,000 rpm the microparticles removed are so small that they can be filtered by the liver, lungs and spleen without causing damage to the body.

A device for rotablation angioplasty is for described for example in U.S. Pat. No. 5,356,418, in EP 0 794 734 B1 and in EP 0 267 539 B1. The device described in EP 0 267 539 B1 for “transluminal microdisection” is known in significant variants as a product of Boston Scientific under the name Rotablator®.

The Rotablator consists of the drill head (appr. 1-3 mm diameter), which is connected via a highly-flexible shaft with a pneumatically driven turbine (typical speed of 20,000-155,000 rpm). The turbine is driven by compressed air and controlled from a console which is activated by a foot pedal.

The flexible shaft consists of a drive cable and is enclosed in a Teflon sleeve through which a rinsing fluid is supplied under pressure. The rinsing fluid on the one hand prevents a heating up of the drive cable, on the other hand it guarantees that the microparticales are washed out to distal. The shaft with the drill head can be changed without the turbine having to be changed. The appr. 3 m long and thin (appr. 0.2-0.3 mm) guide wire (“Rotawire™”), via which the drill probe is advanced is automatically blocked in the turbine during rotablation. This blocking can however be released so that the drill head and the wire can be moved independently of each other. This is frequently used to take the drill head out of the coronary artery.

### SUMMARY OF INVENTION

The therapy described above is undertaken under X-ray control using contrast means with an angiography device. The disadvantage of this method is that the coronary arteries are only shown in two dimensions and only the actual narrowing is shown in the X-ray image. The medical personnel can barely distinguish between plaque and vessel wall during the intervention. The purely angiographical assessment of the seriousness of the calcification and especially the position of the calcium in the plaque (surface vs. deep) is difficult. This involves a significant risk for the patient, either too little plaque is removed and the desired blood flow is not restored or the risk of a restenosis remains, or too much tissue is removed and a perforation of the vessel can occur.

To make the plaque more visible a separate IVUS catheter (Intravascular ultrasound) can be introduced into the vessel. An IVUS system is for example described in DE 198 27 460 A1 and in U.S. Pat. No. 5,193,546.

The disadvantage of this method is that the entire rotablation arrangement must be removed from the vessel each time.

In U.S. Pat. No. 5,312,427 a device is described which has a double-lumen catheter in which one lumen can be used to introduce an IVUS probe. The disadvantage of this solution lies in the double-lumen catheter, of which the diameter must be significantly larger than a catheter normally used and is thus only ill-suited to introduction into coronary arteries. The further disadvantage of this solution lies in the increased stiffness of the catheter resulting from the double lumen required, which makes navigating the catheter in the coronary arteries more difficult. A further disadvantage of this solution lies in the decentralized position of the introduced IVUS probe in relation to the drill head of the rotablator.

An object of the invention is thus to create a device for simplified execution and monitoring of rotablation, in which, without changing catheters, a precise observation of the target area is possible simultaneously with the intervention at the plaque.

To achieve this object there is provision in the invention for a rotablation catheter and an IVUS catheter to be integrated into one unit, with the IVUS line preferably being embodied as a glass fiber line running with the IVUS sensor embodied with the drill head in a highly flexible drive shaft which drives the drill head and the IVUS sensors in a rotating motion.

The inventive combination of an IVUS catheter with a rotablation angioplasty catheter into an integrated unit produces an optimal system for “debulking” coronary arteries. The major advantage of this solution lies in the reduction both of procedural steps and also of the catheters used, as well as in the reduction of the X-ray radiation applied. The images of the IVUS system supply important additional medical information about the plaque and the artery wall, e.g. anti-inflammatory processes. This enables the “blocked” artery section to be better recognized in each case and the removal of the plaque to be checked during and after the procedure.

In accordance with a further feature of the invention there can be provision for a microdrive to be arranged between the drill head and the IVUS sensor, so that the rotation of the drill head can be undertaken independently of the rotation of the OCT sensor. The catheter sleeve can advantageously be provided with entry or exit points in its ends for contrast means or rinsing fluids since it makes sense when using an IVUS catheter to inject a rinsing solution (e.g. physiological salt solution) into the area of the location to be examined.

As well as the arrangement of magnets in the catheter tip for magnetic navigation, an embodiment of the device with a guide wire running through it can be provided.

Finally it is also within the framework of the invention, that at the tip of the catheter an inflatable, preferably multichamber balloon is arranged for fixing the catheter and/or for vessel dilation.

### BRIEF DESCRIPTION OF THE DRAWINGS

Further advantages, features and details of the invention are produced by the subsequent description of an exemplary embodiment as well as by reference to the drawing. The diagrams show:

FIG. 1 a schematic section through an inventive combined IVUS rotablation catheter, in which the IVUS sensor is arranged behind the actual cutting section in the drill head and

FIG. 2 a modified embodiment of such a combined IVUS rotablation catheter with an IVUS sensor arranged to move in front of the drill head.

### DETAILED DESCRIPTION OF INVENTION

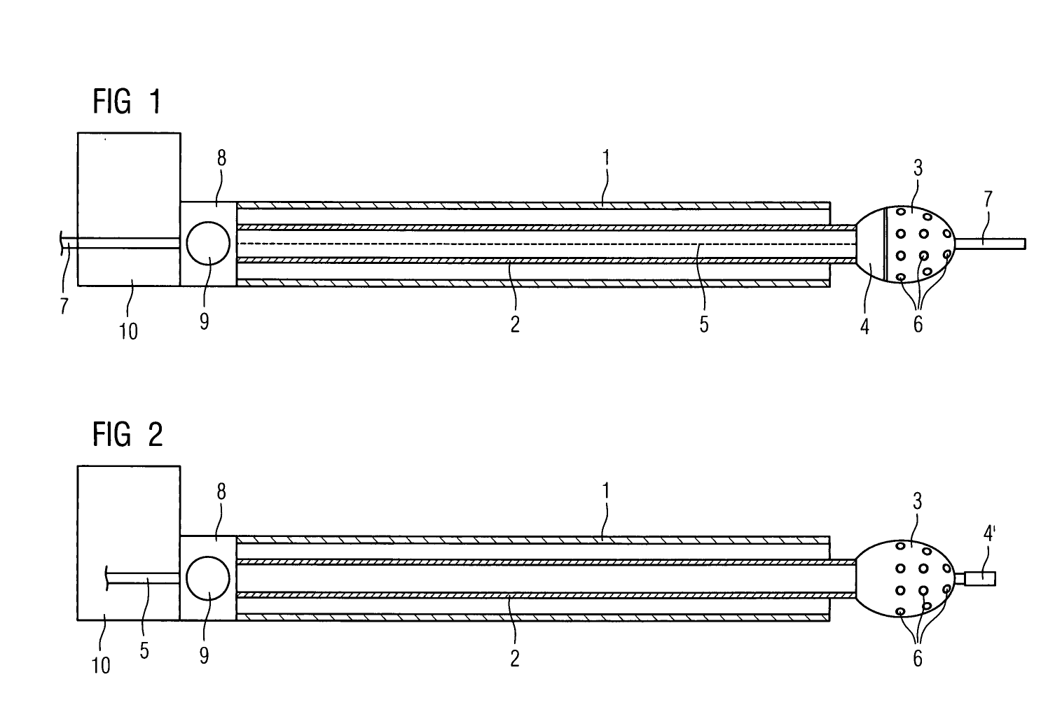

The combined IVUS rotablation catheter shown in FIG. 1 comprises a catheter sheath 1, in which a hollow flexible drive shaft 2 is arranged which is used both to drive the drill head 3 and also to drive the IVUS sensor 4 arranged in its rear section. 5 indicates a glass fiber line forming the signal line to the IVUS sensor 4. The drill head 3 is equipped in the front section with grinding and cutting particles 6 which are embodied so that, as they rotate, they push aside normal vessel tissue and only remove plaque adhering to the inner wall of the vessel. 7 indicates a guide wire which runs through the catheter but for reasons of clarity the middle of said guide wire is not shown which is initially introduced into the vessel to be treated up to the target area before the combination catheter is introduced. Subsequently the inventive combination IVUS rotablation catheter is pushed onto the guide wire and advanced to the target area. Both the guide wire 7 and also the drill head with the integrated IVUS rotablation catheter are introduced in this case under X-ray control, if necessary with contrast means. With the IVUS probe the point at which the plaque is to be removed is examined in more detail (during this examination the combination probe turns a relatively slow speed, for example appr. 100 to 1,500 rpm, with a rinsing fluid being simultaneously injected for the IVUS method. Subsequently the drill head is moved slowly into the stenosis at high speed and after a few seconds is withdrawn slightly. When a specific amount of plaque has been removed the IVUS sensor is used to check the point on the vessel wall. The process is repeated until the plaque is removed from all points.

In addition to the mechanical connection system 8 and the rotation coupling 9 for the connection a signal interface and drive unit 10 is also provided for operation of the combination sensor. In addition the feed lines or outlet lines already discussed are provided for the rinsing fluid, but these are not shown in the diagram in order to aid clarity.

The modified version of the combined IVUS rotablation catheter in accordance with FIG. 2 essentially differs from that shown in FIG. 1 only in that the IVUS sensor is not provided in the drill head behind its cutting particles but is arranged temporarily at 4′ and that the hollow flexible drive shaft 2 is provided with an integrated lumen for guiding the IVUS sensor.

With both embodiments, as well as a magnet in the catheter tip for magnetic navigation a microdrive arranged between the drill head and the IVUS sensors is especially provided to enable the two to be driven at different speeds.

A medical system of combined IVUS rotablation angioplasty catheter, subsystem for connecting the IVUS rotablation angioplasty catheter, consists of the signal Interface unit, preprocessing for IVUS image data, image processing unit and image display unit. In addition there is a user interface for control of the system as well as for operation of the image display for IVUS including image store, power supply unit and network interface (e.g. DICOM), as well as a drive unit for the hollow flexible drive shaft. The drive unit has facilities for providing the high speed (e.g. 150,000 rpm) for the drill head and also the low speed (appr. 1,000 rpm) for the IVUS probe. At the low speed for the IVUS probe a relatively constant speed is necessary so that expediently the high speed is generated in the known way with the compressed air turbine, while the low speed can be created with a regulated electronic drive.

The IVUS image system can be expanded by menus, to allow quantification (e.g. measurement of the angles, lengths, services, degree of stenosis before and after the procedure) of the stenosis and of the removed plaque.

Finally it would also still be possible—as well as using normal X-ray markers on the catheter shaft—to fit a temperature sensor at the tip of the catheter which is not shown in the drawing of the exemplary embodiment, to check the heat produced by the friction at high speeds. In clinical studies it has namely been shown that heat damage in the vessels increases the residual oserate.

1-8. (canceled)

9. A device for applying and monitoring medical rotablation, comprising:
a rotablation catheter device;
an IVUS catheter device having an IVUS sensor and an IVUS signaling line connected to the IVUS sensor for monitoring the rotablation, the rotablation catheter device and the IVUS catheter device integrated into one catheter unit having a catheter tip; and
a rotating drill head arranged at the catheter tip for removing vascular plaque, the drill head sized and configured to deflect normal vascular tissue while removing plaque from a vascular wall affected by plaque, wherein the drill head is operatively connected to the IVUS sensor.

10. The device according to claim 9, further comprising a hollow, flexible drive shaft for rotating the drill head and the IVUS sensor, wherein the IVUS signaling line is arranged within the drive shaft.

11. The device according to claim 11, wherein the IVUS signaling line comprises an optical fiber.

12. The device according to claim 9, further comprising a micro gear unit for connecting the drill head to the IVUS sensor.

13. The device according to claim 9, further comprising a catheter jacket for accommodating the catheter unit, the catheter jacket having inlet or outlet openings for feeding to respectively discharging from the catheter unit a contrast medium or a rinsing fluid.

14. The device according to claim 9, further comprising a plurality of magnets arranged at the catheter tip for magnetic navigation of the catheter unit.

15. The device according to claim 9, further comprising a continuous guide wire.

16. The device according to claim 9, further comprising an inflatable balloon arranged at the catheter tip for locating the catheter device or dilating a vessel.

17. The device according to claim 16, wherein the balloon comprises a plurality of inflatable chambers.

18. The device according to claim 9, further comprising a temperature sensor arranged at the catheter tip.
